(19) United States
(12) Patent Application Publication
(10) Pub. No.: US 2009/0042764 A1
(43) Pub. Date: Feb. 12, 2009

(54) Cleaning Agent Comprising Complexes with Bleach Catalytic Activity

(75) Inventors: Pavel Gentschev, Monheim (DE); Arndt Kessler, Monheim Am Rhein (DE); Maren Jekel, Willich (DE); Johannes Zipfel, Dusseldorf (DE); Christian Nitsch, Dusseldorf (DE); Doris Dahlmann, Dusseldorf (DE); Steve Doring, Mettmann (DE)

Correspondence Address: CONNOLLY BOVE LODGE & HUTZ LLP, PO BOX 2207, WILMINGTON, DE, 19899-2207, US

(73) Assignee: Henkel AG & Co. KGaA, Dusseldorf (DE)

(21) Appl. No.: 12/159,639

(22) PCT Filed: Dec. 8, 2006
(86) PCT No.: PCT/EP2006/011817

§ 371 Date: Aug. 7, 2008

(30) Foreign Application Priority Data
Dec. 29, 2005 (DE) 10 2005 063 059.6

(51) Int. Cl. C11D 3/28; C07D 255/00

(52) U.S. Cl. 510/500; 540/472; 540/465

(57) ABSTRACT

Hard surface cleaning agents comprising a macrocyclic compound of the general formula L:

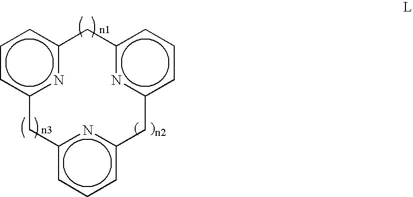

wherein each of n1, n2 and n3 independently represents 1 or 2; and hard surface cleaning agents comprising a transition metal complex of the general formula (I):

[MLₐX_{b}]cY (1)

wherein M represents a metal selected from the group consisting of manganese, iron, cobalt, copper, ruthenium, molybdenum, and combinations thereof; X represent a neutral or anionic ligand; Y represents a non-complex-bound anion; a represents 1 or 2; each of b and c independently represents a number of 0 to 6, with the proviso that b and c are selected such that the complex is neutral based on M, X and Y; and L represents a macrocyclic ligand of the general formula L; and methods of cleaning hard surfaces therewith.

The present invention relates to the use of certain transition-metal complexes as catalytically active activators for, in particular, inorganic peroxygen compounds for bleaching colored stains on hard surfaces and cleaning agents for hard surfaces that contain such catalysts.

Inorganic peroxygen compounds, in particular hydrogen peroxide, and solid peroxygen compounds that dissolve in water with the release of hydrogen peroxide, such as sodium perborate and sodium carbonate perhydrate, have been used for a long time as oxidizing agents for disinfecting and bleaching purposes. The oxidizing effect of these substances in dilute solutions depends greatly on temperature; with H₂O₂ or perborate in alkaline bleaching baths, for example, sufficiently rapid bleaching of stained textiles is achieved only at temperatures above approximately 80° C. At lower temperatures, the oxidizing effect of the inorganic peroxygen compounds can be improved by adding so-called bleach activators, for which a number of proposals have been disclosed in the literature, chiefly from the substance classes of the N- or O-acyl compounds, for example multiply acylated alkylenediamines, in particular tetraacetylethylenediamine, acylated glycourils, in particular tetraacetyl glycouril, N-acylated hydantoins, hydrazides, triazoles, hydrotriazines, urazoles, diketopiperazines, sulfurylamides, and cyanurates; also carboxylic acid anhydrides, in particular phthalic acid anhydride, carboxylic acid esters, in particular sodium nonanoyloxybenzenesulfonate, sodium isononanoyloxybenzenesulfonate, and acylated sugar derivatives such as pentaacetyl glucose. By adding these substances, the bleaching effect of aqueous peroxide baths can be enhanced sufficiently that at temperatures of only about 60° C., substantially the same effects are obtained as with the peroxide bath alone at 95° C.

Even these temperatures are too high for manual cleaning of hard surfaces, for example tableware, and are not normally reached even in automatic dishwashing methods. As efforts are made toward energy-saving methods for automatic cleaning of tableware, application temperatures below 60° C., in particular below 50° C. and as low as cold-water temperature, have become increasingly important in recent years.

At these low temperatures, the effect of the previously known activator compounds generally declines perceptibly, particularly for hard-to-bleach stains such as, for example, tea residues on china or glass. There has therefore been no lack of efforts to develop more-effective activators for this temperature range, although until now no convincing success has been achieved. One starting point for this might result from the use of transition-metal salts and complexes as so-called bleach catalysts. European Patent Application EP 630 964, for example, discloses specific manganese complexes of the salen type that, as indicated therein, have no pronounced effect in terms of bleach intensification of peroxygen compounds, and can only produce bleaching of dirt or coloring matter that is present in washing baths, i.e. has already been detached from the textile surface to be cleaned.

Surprisingly, it has now been found that transition-metal complexes having a macrocyclic ligand that comprises three pyridine groups have a definite bleach-catalyzing effect on colored stains that are present on hard surfaces.

A subject of the invention is therefore the use of transition-metal complexes of formula (I)

[MLₐX_{b}]cY (I)

in which

M denotes manganese, iron, cobalt, copper, ruthenium, or molybdenum,

L denotes the macrocyclic ligand

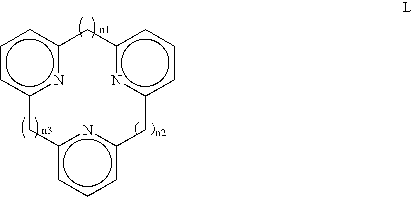

in which n1, n2, and n3, mutually independently, are 1 or 2,
X denotes a neutral or anionic ligand,
Y denotes a non-complex-bound anion,
a denotes 1 or 2, and
b and c mutually independently, denote numbers from 0 to 6, with the stipulation that their sum is to be selected to yield neutrality for the compound as a function of the charge of the central atom M and the charges of X and Y,
   as activators for, in particular, inorganic peroxygen compounds in cleaning solutions for hard surfaces, in particular for tableware.

A preferred transition metal (M in formula I) is manganese.

In the compounds L, the pyridine rings can be bridged with three ethylene units (n1=n2=n3=2); by preference, two ethylene units and one methylene unit (n1=n2=2, n3=1), or one ethylene unit and two methylene units (n1=2, n2=n3=1), and particularly preferably three methylene units (n1=n2=n3 =1) are present there.

Production of the macrocyclic ligand in formula (I), where n1=n2=n3=1, can be accomplished by the following steps, which can be applied analogously for different values of the indices n1, n2, and/or n3:

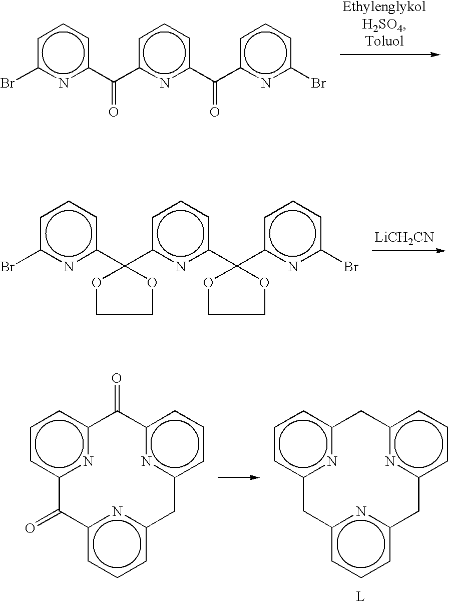

Neutral ligands X in the complex compounds of formula (I) can be, for example, water or ammonia. An anionic ligand X that is, if applicable, charge-equalizing together with non-complex-bound anions Y in the compounds of formula (I) can, like anion Y, be monovalent or polyvalent. X and Y are preferably a halide, in particular chloride, hydroxide, hexafluorophosphate, perchlorate, an oxo, peroxo, or hydroperoxo anion, or the anion of a carboxylic acid, such as formate, acetate, benzoate, or citrate. Anions Y and/or anion ligands X are present in a quantity (c or b in formula I) such that the compound as a whole according to formula (I) has no charge.

A further subject of the invention is corresponding use of the macrocyclic compound L in the presence of a salt of manganese, iron, cobalt, copper, ruthenium, or molybdenum in order to intensify the bleaching performance of, in particular, inorganic peroxygen compounds in cleaning solutions for hard surfaces. In the presence of water, a complex of formula (I) presumably forms from the metal salt and compound L.

The invention further relates to cleaning agents for hard surfaces, in particular cleaning agents for tableware, and thereamong by preference those for use in automatic cleaning methods, that contain a macrocyclic compound L

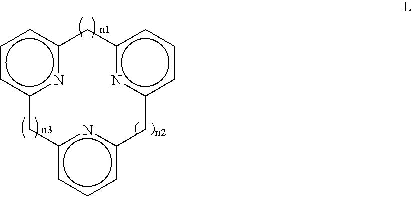

in which n1 n2, and n3, mutually independently, are 1 or 2. Such agents also, by preference, contain a salt of manganese, iron, cobalt, copper, ruthenium, or molybdenum, or they are used in the presence of such metal salts, which are added separately or if applicable derive from the water that is used, so that a bleach-catalyzing complex can form from them and from ligand L. Bleach-catalyzing complexes having ligand L can exert their effect in the presence of atmospheric oxygen, so that agents according to the present invention can also be free of oxidizing agents. By preference, however, they contain a bleaching agent described in further detail below, or such an agent is added separately in the context of a cleaning method in which an agent according to the present invention is utilized.

The use according to the present invention substantially comprises the creation, in the presence of a hard surface contaminated with colored stains, of conditions in which an oxidizing agent and the bleach catalyst according to formula (I) can react with one another, with the goal of obtaining subsequent products that act in more strongly oxidizing fashion. Such conditions exist, in particular, when the two reaction partners encounter one another in aqueous solution. This can occur as a result of separate addition of the peroxygen compound and the bleach catalyst to a solution that, if applicable, contains cleaning agent. Particularly advantageously, however, the method according to the present invention is carried out using a cleaning agent according to the present invention for hard surfaces that contains the bleach catalyst and, if applicable, contains a peroxygen-containing oxidizing agent. The peroxygen compound can also be added separately to the solution, in substance or as a preferably aqueous solution or suspension, if a peroxide-free cleaning agent is used. It is also possible, if desired, to omit the addition of the peroxygen compound if the method is carried out in the presence of gaseous oxygen, for example from air.

The conditions can be greatly varied depending on the intended use. For example, in addition to purely aqueous solutions, mixtures of water and suitable organic solvents are also possible as a reaction medium. The quantities of peroxygen compounds used are preferably selected so that from 10 ppm to 10% active oxygen, in particular from 50 ppm to 5000 ppm active oxygen, is present in the solutions. The quantity of bleach catalyst used also depends on the intended application. Depending on the desired degree of activation, 0.00001 mol to 0.025 mol, by preference 0.001 mol to 0.02 mol, catalyst per mol of peroxygen compound is used, but the value can also exceed or fall below these limits in particular cases.

A further subject of the invention is a cleaning agent for hard surfaces, in particular for tableware, that contains 0.001 wt % to 1 wt %, in particular 0.005 wt % to 0.1 wt %, of a bleach catalyst according to formula (i), in addition to usual ingredients compatible with the bleach catalyst. The bleach catalyst can, in a manner known in principle, be adsorbed onto carrier substances and/or be embedded into encasing substances.

The invention additionally relates to corresponding methods for cleaning hard surfaces, in particular tableware, using a ligand L or a bleach catalyst of formula (I), respectively.

The cleaning agents according to the present invention, which can be present as powdered or tabletted solids, homogeneous solutions, or suspensions, can in principle contain, in addition to the bleach catalyst used according to the present invention, all known ingredients that are usual in such agents. The agents according to the present invention can contain, in particular, builder substances, surface-active surfactants, peroxygen compounds, water-miscible organic solvents, enzymes, sequestering agents, electrolytes, pH regulators, and further adjuvants such as silver corrosion inhibitors, foam regulators, additional peroxygen activators, and coloring agents and fragrances. Agents according to the present invention provided for use in automatic dishwashing methods are preferably so-called “3 in 1” products that combine the conventional cleaner, rinse-agent, and regenerating-salt agents in one agent.

A cleaning agent according to the present invention for hard surfaces can furthermore contain abrasively acting constituents, in particular from the group comprising quartz flour, wood flour, plastic flour, chalks, and glass microspheres, as well as mixtures thereof. Abrasive substances are contained in the cleaning agents according to the present invention by preference at no more than 20 wt %, in particular from 5 wt % to 15 wt %.

A further subject of the invention is a low-alkalinity agent for automatic cleaning of tableware, a 1-wt % solution of which agent has a pH from 8 to 11.5, by preference 9 to 10.5, containing 15 wt % to 60 wt %, in particular 30 wt % to 50 wt %, water-soluble builder component, 5 wt % to 25 wt %, in particular 10 wt % to 15 wt %, oxygen-based bleaching agent, based in each case on the entire agent that contains a bleach catalyst according to formula (I), in particular in quantities from 0.005 wt % to 0.1 wt %. A particularly preferred embodiment of the invention is an automatic dishwashing agent that is present in the form of a tablet, by preference in the form of a multi-phase tablet in which the phases have different contents of bleach catalyst according to formula (I) and of ligand molecule L. A “different content” means in this context, for example, that one phase of the tablet contains the entirety, or at least the greatly predominant portion, of the bleach catalyst according to formula (I), and another phase contains the entirety, or at least the greatly predominant portion, of the oxygen-based bleaching agent. It is also possible, however, to incorporate the entirety, or at least the greatly predominant portion, of both the oxygen-based bleaching agent and the bleach catalyst according to formula (I) together into one phase of the agent in tablet form, and to introduce into the other, or at least another, phase of the tablet the entirety, or at least the greatly predominant portion, of the bleach-sensitive active substances, for example enzymes. Instead of the already-preformed complex of formula (I), ligand molecule L can also be present if a metal salt additionally is present or is added, so that the complex of formula (I) can form under utilization conditions.

Suitable in principle as water-soluble builder components, in particular in the low-alkalinity cleaning agents, are all builders usually used in automatic dishwashing agents, for example polymeric alkali phosphates, which can be present in the form of their alkaline, neutral, or acid sodium or potassium salts. Examples thereof are tetrasodium diphosphate, disodium dihydrogendiphosphate, pentasodium triphosphate, so-called sodium hexametaphosphate, and the corresponding potassium salts, or mixtures of sodium and potassium salts. Their quantities can be in the range of up to approximately 35 wt %, based on the entire agent; preferably, however, the agents according to the present invention are free of such phosphates. Further possible water-soluble builder components are, for example, organic polymers or natural or synthetic origin, chiefly polycarboxylates, which act as co-builders especially in hard-water regions. Possibilities are, for example, polyacrylic acids and copolymers of maleic acid anhydride and acrylic acid, as well as the sodium salts of said polymeric acids. Commercially usual products are, for example, Sokalan® CP 5 and PA 30 of the BASF company. Among the polymers of natural origin usable as co-builders are, for example, oxidized starch as known, for example, from International Patent Application WO 94/05762, and polyamino acids such as polyglutamic acid or polyaspartic acid. Further possible builder components are naturally occurring hydroxycarboxylic acids such as, for example, mono-, dihydroxysuccinic acid, α-hydroxypropionic acid, and gluconic acid. Among the preferred builder components are the salts of citric acid, in particular sodium citrate. Possibilities as sodium citrate are anhydrous trisodium citrate and, by preference, trisodium citrate dihydrate. Trisodium citrate dihydrate can be used as a finely or coarsely crystalline powder. Depending on the pH ultimately established in the agents according to the present invention, the acids corresponding to the aforesaid co-builder salts can also be present.

Suitable oxygen-based bleaching agents are principally alkali perborate mono- or tetrahydrate and/or alkali percarbonate, sodium being the preferred alkali metal. The use of sodium percarbonate has advantages especially in cleaning agents for tableware, since it has particularly favorable effects on corrosion properties on glassware. The oxygen-based bleaching agent is therefore by preference an alkali percarbonate, in particular sodium percarbonate. Additionally or, in particular, alternatively, known peroxycarboxylic acids, for example dodecanediperacid or phthalimidopercarboxylic acids, which if applicable can be substituted on the aromatic portion, can be contained. The addition of small quantities of known bleaching-agent stabilizers such as, for example, phosphonates, borates or metaborates, and metasilicates, as well as magnesium salts such as magnesium sulfate, may furthermore be expedient. Because cleaning agents are usually utilized in an air atmosphere, the agents according to the present invention can also be free of bleaching agents, since the bleach catalysts according to formula (I) already exhibit an effect in the presence of atmospheric oxygen.

In addition to the bleach catalysts according to formula (I), further transition-metal salts or complexes known as bleach-activating active substances, and/or conventional bleach activators, i.e. compounds that, under perhydrolysis conditions, yield optionally substituted perbenzoic acid and/or peroxycarboxylic acids having 1 to 10 carbon atoms, in particular 2 to 4 carbon atoms, can be used; the presence of peroxygen-based bleaching agent is then necessary, however. The usual bleach activators cited above, which carry O-and/or N-acyl groups having the aforesaid number of carbon atoms, and/or which carry optionally substituted benzoyl groups, are suitable. Multiply acylated alkylenediamines, in particular tetraacetylethylendiamine (TAED), acylated glycolurils, in particular tetraacetyl glycoluril (TAGU), acylated triazine derivatives, in particular 1,5-diacetyl-2,4-dioxohexahydro-1,3,5-triazine (DADHT), acylated phenylsulfonates, in particular nonanoyl- or isononanoyloxybenzenesulfonate, acylated polyvalent alcohols, in particular triacetin, ethylene glycol diacetate, 2,5-diacetoxy-2,5-dihydrofuran, as well as acetylated sorbitol and mannitol, and acylated sugar derivatives, in particular pentaacetyl glucose (PAG), pentaacetyl fructose, tetraacetyl xylose, and octaacetyl lactose, as well as acetylated, optionally N-alkylated glucamine and gluconolactone, are preferred. Combinations of conventional bleach activators can also be used.

Automatic dishwashing agents according to the present invention preferably contain the usual alkali carriers such as, for example, alkali silicates, alkali carbonates, and/or alkali hydrogencarbonates. Included among the alkali carriers usually used are carbonates, hydrogencarbonates, and alkali silicates having a SiO₂/M₂O molar ratio (M=alkali atom) from 1.5:1 to 2.5:1. Alkali silicates can be contained in quantities of up to 30 wt % based on the entire agent. It is preferable to dispense entirely with the use of the highly alkaline metasilicates as alkali carriers. The alkali carrier system preferably used in the agents according to the present invention is a mixture of carbonate and hydrogencarbonate, by preference sodium carbonate and hydrogencarbonate, which is contained in a quantity of up to 60 wt %, by preference 10 wt % to 40 wt %. The ratio of carbonate used to hydrogencarbonate used varies depending on the pH that is ultimately desired, although an excess of sodium hydrogencarbonate is usually used, so that the weight ratio between hydrogencarbonate and carbonate is generally 1:1 to 15:1.

In a further preferred embodiment of agents according to the present invention, 20 wt % to 40 wt % water-soluble organic builders, in particular alkali citrate, 5 wt % to 15 wt % alkali carbonate, and 20 wt % to 40 wt % alkali disilicate are contained.

The agents according to the present invention can also, if applicable, have surfactants added to them, in particular low-foaming nonionic surfactants that provide better detachment of grease-containing stains and serve as a wetting agent and, if applicable, as a granulating adjuvant in the context of manufacture of the cleaning agents. Their quantity can be up to 10 wt %, in particular up to 5 wt %, and is preferably in the range from 0.5 wt % to 3 wt %. Extremely low-foaming compounds are usually used, especially in cleaning agents for use in automatic dishwashing processes. These compounds include, by preference, C12-C18 alkylpolyethylene glycol-polypropylene glycol ethers having respectively up to 8 mol of ethylene oxide and propylene oxide units in the molecule. It is also possible, however, to use other known low-foaming nonionic surfactants such as, for example, C₁₂-C₁₈ alkylpolyethylene glycol-polybutylene glycol ethers having respectively up to 8 mol of ethylene oxide and butylene oxide units in the molecule, end-capped alkylpolyalkylene glycol mixed ethers, and the foaming but environmentally attractive C₈-C₁₄ alkyl polyglucosides having a degree of polymerization from approximately 1 to 4 (e.g. APG® 225 and APG® of the Henkel company), and/or C₁₂-C₁₄ alkylpolyethylene glycols having 3 to 8 ethylene oxide units in the molecule. Likewise suitable are surfactants from the family of the glucamides such as, for example, alkyl-N-methylglucamides, in which the alkyl part preferably derives from a fatty alcohol having a carbon chain length of C₆ to C₁₄. It is in some cases advantageous if the above-described surfactants are used as mixtures, for example the combination of alkyl polyglycoside with fatty alcohol ethoxylates, or glucamide with alkyl polyglycosides.

Although it is known that transition-metal complexes, in particular manganese complexes, can counteract the corrosion of silver, the compounds of formula (I) are usually used in quantities that are too small to bring about silver corrosion protection, so that it is additionally possible to use, in cleaning agents according to the present invention for tableware, silver corrosion inhibitors whose action can be intensified by the compounds according to formula (I). Preferred silver corrosion protection agents are organic disulfides, divalent phenols, trivalent phenols, optionally substituted benzotriazole, salts and/or complexes of manganese, titanium, zirconium, hafnium, vanadium, cobalt, or cerium, in which the aforesaid metals are present in one of the oxidations states II, III, IV, V, or VI.

In addition, the agents according to the present invention can contain enzymes such as proteases, amylases, pullulases, cutinases, and lipases, for example proteases such as BLAP®, Optimase®, Opticlean®, Maxacal®, Maxapem®, Esperase®, and/or Savinase®, amylases such as Termamyl®, Amylase-LT®, Maxamyl®, and/or Duramyl®, lipases such as Lipolase®, Lipomax®, Lumafast®, and/or Lipozym®. The enzymes that are used if applicable can be adsorbed onto carrier substances and/or embedded into encasing substances in order to protect them from premature inactivation. They are contained in the cleaning agents according to the present invention in quantities by preference not above 2 wt %, in particular from 0.1 wt % to 0.7 wt %.

If the cleaning agents foam excessively upon use, they can also have added to them up to 6 wt %, by preference approximately 0.5 wt % to 4 wt %, of a foam-suppressing compound, by preference from the group of the silicone oils, mixtures of silicone oil and hydrophobized silicic acid, paraffins, paraffin-alcohol combinations, hydrophobized silicic acid, the bis-fatty acid amides, and other known defoamers obtainable commercially. Further optional ingredients in the agents according to the present invention are, for example, perfume oils.

Among the organic solvents usable in the agents according to the present invention, especially when the latter are present in liquid or paste form, are alcohols having 1 to 4 carbon atoms, in particular methanol, ethanol, isopropanol, and tert.-butanol, diols having 2 to 4 carbon atoms, in particular ethylene glycol and propylene glycol, as well as mixtures thereof, and the ethers derivable from the aforesaid compound classes. Water-miscible solvents of this kind are present in the cleaning agents according to the present invention at preferably no more than 20 wt %, in particular from 1 wt % to 15 wt %.

In order to establish a desired pH that does not of itself result from the mixture of the other components, the agents according to the present invention can contain system-compatible and environmentally compatible acids, for example citric acid, acetic acid, tartaric acid, malic acid, lactic acid, glycolic acid, succinic acid, glutaric acid, and/or adipic acid, but also mineral acids, in particular sulfuric acid, or alkali hydrogensulfates or bases, in particular ammonium hydroxides or alkali hydroxides pH regulators of this kind are contained in the agents according to the present invention by preference at no more than 10 wt %, in particular from 0.5 wt % to 6 wt %.

Manufacture of the solid agents according to the present invention presents no difficulties and can in principle be effected in known fashion, for example by spray drying or granulation, the peroxygen compound and bleach catalyst optionally being separately added later. If applicable, the bleach catalyst is mixed with further raw materials and/or compounds, and the mixture is then compressed into tablets or phases thereof.

Cleaning agents according to the present invention in the form of aqueous solutions or those containing other usual solvents are manufactured particularly advantageously by simply mixing the ingredients, which can be placed, in substance or as a solution, into an automatic mixer.

The agents according to the present invention are present by preference as powdered, granular, or tabletted preparations that can be manufactured in known fashion, for example by mixing, granulating, roller compacting, and/or by spray drying of the thermally insensitive components and mixing in the more-sensitive components, included among which are, in particular, enzymes, bleaching agents, and the bleach catalyst.

For the manufacture of cleaning agents according to the present invention in tablet form, it is preferable to proceed in such a way that all the constituents, or all the constituents provided for use together in one phase of the tablet, are mixed together in a mixer, and the mixture, or the mixtures in succession, are compressed by means of conventional tablet presses, for example eccentric presses or rotary tablet presses, at compression pressures in the range from 200·10⁵ Pa to 1500·10⁵ Pa. Break-resistant tablets that are nevertheless sufficiently rapidly soluble under the utilization conditions, having flexural strength values normally above 150 N, are thereby obtained without difficulty. A tablet manufactured in this fashion preferably has a weight from 15 g to 40 g, in particular from 20 g to 30 g. It can be round, for example with a diameter from 35 mm to 40 mm, or can have any other desired shape, for example rectangular with optionally rounded edges.

Agents according to the present invention in the form of non-dusting, shelf-stable, pourable powders and/or granulates having high bulk densities in the range from 800 to 1000 g/l can be manufactured by mixing the builder components with at least a portion of liquid mixture components in a first partial step of the method, with an increase in the bulk density of this premixture, and then, if desired after intervening drying, combining the further constituents of the agent, among them the bleach catalyst, with the premixture thus obtained.

Agents according to the present invention for cleaning tableware can be used in both household and commercial dishwashers. They are added manually or by means of suitable metering devices The utilization concentration in the cleaning bath is as a rule approximately 1 to 8 g/l, by preference 2 to 5 g/l.

An automatic washing cycle is generally supplemented and completed by several intermediate rinse cycles with clean water following the cleaning cycle, and a rinse cycle with an ordinary rinse agent. When agents according to the present invention are used, tableware that is completely clean and unobjectionable in hygienic terms is obtained after drying.

1-12. (canceled)

13. A hard surface cleaning agent comprising a macrocyclic compound of the general formula L: 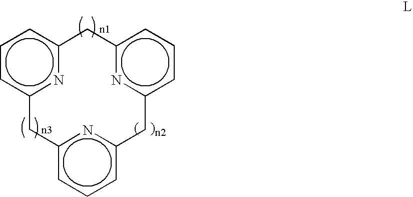
wherein each of n1, n2 and n3 independently represents 1 or 2.

14. The hard surface cleaning agent according to claim 13, further comprising a salt of a metal selected from the group consisting of manganese, iron, cobalt, copper, ruthenium, molybdenum and mixtures thereof.

15. The hard surface cleaning agent according to claim 13, further comprising a peroxygen compound.

16. The hard surface cleaning agent according to claim 15, wherein the peroxygen compound comprises one or more selected from the group consisting of organic peracids, hydrogen peroxide, perborates, percarbonates, and mixtures thereof.

17. A hard surface cleaning agent comprising a transition metal complex of the general formula (I):
[MLₐX_{b}]cY (I)
wherein M represents a metal selected from the group consisting of manganese, iron, cobalt, copper, ruthenium, molybdenum, and combinations thereof; X represent a neutral or anionic ligand; Y represents a non-complex-bound anion; a represents 1 or 2; each of b and c independently represents a number of 0 to 6, with the proviso that b and c are selected such that the complex is neutral based on M, X and Y; and L represents a macrocyclic ligand of the general formula L: 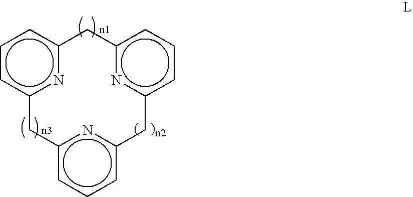
wherein each of n1, n2 and n3 independently represents 1 or 2.

18. The hard surface cleaning agent according to claim 17, wherein M represents manganese.

19. The hard surface cleaning agent according to claim 17, wherein the transition metal complex of the general formula (I) is present in an amount of 0.001 to 1% by weight.

20. The hard surface cleaning agent according to claim 18, wherein the transition metal complex of the general formula (I) is present in an amount of 0.001 to 1% by weight.

21. The hard surface cleaning agent according to claim 17, wherein the transition metal complex of the general formula (I) is present in an amount of 0.005 to 0.1% by weight.

22. The hard surface cleaning agent according to claim 18, wherein the transition metal complex of the general formula (I) is present in an amount of 0.005 to 0.1% by weight.

23. The hard surface cleaning agent according to claim 16, further comprising a peroxygen compound.

24. The hard surface cleaning agent according to claim 22, further comprising a peroxygen compound.

25. The hard surface cleaning agent according to claim 23, wherein the peroxygen compound comprises one or more selected from the group consisting of organic peracids, hydrogen peroxide, perborates, percarbonates, and mixtures thereof.

26. The hard surface cleaning agent according to claim 23, wherein the peroxygen compound comprises one or more selected from the group consisting of alkali perborate monohydrates, alkali perborate tetrahydrates, alkali percarbonates, peroxycarboxylic acids, and mixture thereof.

27. A method comprising: (a) providing a hard surface cleaning agent according to claim 13; and (b) applying the hard surface cleaning agent to a hard surface to be cleaned.

28. A method comprising: (a) providing a hard surface cleaning agent according to claim 14; and (b) applying the hard surface cleaning agent to a hard surface to be cleaned.

29. A method comprising: (a) providing a hard surface cleaning agent according to claim 15; and (b) applying the hard surface leaning agent to a hard surface to be cleaned.

30. A method comprising: (a) providing a hard surface cleaning agent according to claim 17; and (b) applying the hard surface cleaning agent to a hard surface to be cleaned.

31. A method comprising: (a) providing a hard surface cleaning agent according to claim 19; and (b) applying the hard surface cleaning agent to a hard surface to be cleaned.

32. A method comprising: (a) providing a hard surface cleaning agent according to claim 20; and (b) applying the hard surface cleaning agent to a hard surface to be cleaned.
